(19) United States
(12) Patent Application Publication
(10) Pub. No.: US 2026/0315343 A1
(43) Pub. Date: Oct. 15, 2026

(54) LIP BALM CONTAINING SUNSCREEN

(71) Applicant: CASTOR BRANDS COMPANY LLC, Hackensack, NJ (US)

(72) Inventor: Angela Eppler, Richmond, VA (US)

(21) Appl. No.: 19/116,602

(22) PCT Filed: Oct. 4, 2023
(86) PCT No.: PCT/US2023/075945

§ 371 Date: Mar. 28, 2025

Related U.S. Application Data
(60) Provisional application No. 63/378,806, filed on Oct. 7, 2022.

(51) Int. Cl. A61K 8/27; A61K 8/92; A61Q 17/04; A61Q 19/00

(52) U.S. Cl. CPC A61K 8/27; A61K 8/922; A61Q 17/04; A61Q 19/001; A61K 2800/30; A61K 2800/651; A61K 2800/87

(57) ABSTRACT

A composition comprising lip balm base and a sunscreen is provided. The sunscreen can be dispersed within the lip balm base. The composition can have a solid stick structure.

### FIELD OF THE INVENTION

A stick lip balm comprising a physical sunscreen is provided.

### BACKGROUND TO THE INVENTION

Exposure to the sun and its ultraviolet radiation can cause damage to and premature aging of the skin, including the skin covering your lips. Such exposure can cause the lips to become discolored, cracked, and dry. The lip skin may also develop sores, ulcers, or blisters.

A lip balm can contain sunscreen to help protect its skin from damage due to exposure to the sun and particularly its ultraviolet radiation. Chemical sunscreen is a type of sunscreen that contains an active ingredient that absorbs the radiation, sacrificing itself instead of the radiation reaching the skin. Some chemical sunscreens have been found to cause harmful side effects both in humans and for the environment.

Physical sunscreen, on the other hand, contains an active ingredient that sits on the skin and acts as a physical barrier reflecting the radiation away from the skin, preventing the radiation from reaching the skin. While physical sunscreen (also known as mineral sunscreen) can be safer, it is also known to be chalky and difficult to spread. Physical sunscreen can also dry out the skin and leave behind an opaque white cast.

It would be desirable for a sunscreen that can help protect the lip skin from damage due to ultraviolet rays, maintain lip skin moisturization, and prevent drying, chapping and cracking of lip skin, all while avoiding the potentially harmful side effects of chemical sunscreen.

### SUMMARY OF THE INVENTION

A composition comprising lip balm base and a sunscreen dispersed within the lip balm base, wherein the composition is a lip balm composition having a solid stick structure. A composition comprising a lip balm base and a sunscreen dispersed within the lip balm base, wherein the lip balm composition maintains moisturization on a lip of a user for at least 8 hours after it is applied to the lip of the user. A composition comprising a lip balm base and a sunscreen dispersed within the lip balm base, wherein, when applied to a user, the lip balm composition does not leave an opaque white cast on a lip of the user.

### DETAILED DESCRIPTION OF THE INVENTION

### Definitions

A lip balm composition with efficacious amounts of a physical sunscreen and organoleptic/sensory attributes of lip feel associated with moisturizers and emollients is provided. The lip balm of this disclosure can be formed into a stick sufficiently robust to substantially retain the stick shape under normal conditions of shipping, storage, and consumer usage.

As used herein, a “botanical butter” is a solid, meaning it is solid at room temperature, fat and/or oil extract of a plant material such as a fruit and/or seed characterized by having emollient properties.

As used herein, an oil is a fat and/or oil extract that is liquid at room temperature and is characterized by having emollient properties. A “botanical oil” is an oil that is an extract of a plant material such as a fruit and/or seed. A botanical oil can include both pure extracts from a plant fruit or seed and/or extract from a plant fruit or seed combined with additional lipid material to achieve the melting point characteristic and/or lubricity. Preferably the lipid material is derived from a botanical source.

As used herein, a “botanical lipid ester” is lipid ester derived from a botanical source. For example, a jojoba ester is a lipid ester derived from a jojoba plant. Other lipid esters of different botanical origin may be included.

As used herein, a “natural wax” is a wax that occurs naturally or is naturally sourced and is not derived from petroleum. A natural wax can include a mammalian wax and a conventional wax. As used herein, a “mammalian wax” is a wax that is derived from a mammal. As used herein, a “conventional wax” or a “non-mammalian wax” is a wax that is derived from non-mammalian sources such as an insect or a plant. A non-limiting list of a non-mammalian wax can include candelilla wax, carnauba wax, and beeswax. As used herein, a “botanical wax” is a form of conventional wax that is derived from a plant. As used herein, a “synthetic wax” is a wax that is not derived from natural sources, such as a petroleum wax.

As used herein, a “nano material” is a material where the particles of the material have an average size distribution of less than 100 nanometers, typically 10 to 20 nanometers, whereas a “non-nano material” is a material where the particles of the material have average size distribution of greater than 100 nanometers.

The terms “about” or “approximately” mean within an acceptable range for the particular parameter specified as determined by one of ordinary skill in the art, which will depend, in part, on how the value is measured or determined, e.g., the limitations of the measurement system. For example, “about” can mean a range of up to 10% of a given value.

“Percent” or “%” as used herein refers to the percentage by weight of the total composition, unless otherwise specified.

An “emollient” is a cosmetic ingredient which can help maintain or impart a soft, smooth and pliable appearance of skin tissue. Typically, emollients impart sensory properties of smoothness and/or softness when the composition is applied to the lips.

A “lip balm” is a semisolid composition for application to the lips that has protective and/or moisturizing properties. Optionally, lip balms may contain medicaments and/or ingredients that promote lip health such as, for example, sunscreens. Lip balms may be colored but unlike lipsticks do not primarily impart an opaque color to the lips.

A “stick lip balm” or a “lip balm having a solid stick structure” is a lip balm formed into a stick shape that is typically dispensed from a container that permits extension of the lip balm stick from the container and retraction of the lip balm stick back into the container.

As used herein, a “chemical sunscreen” is a material containing active ingredients that absorb UV radiation before it is absorbed by the skin. Chemical sunscreens include materials such as oxybenzone, avobenzone, octisalate, octocrylene, homosalate, and octinoxate. Aminobenzoic acid and trolamine salicylate also operate as chemical sunscreens and are included under this definition even though they are not approved by the US Food and Drug Administration to be listed as a chemical sunscreen. A “physical sunscreen” or “mineral sunscreen” is a material that works by creating a physical barrier on the skin that reflects UV radiation away from the skin. Physical sunscreens include, for example, zinc oxide and titanium dioxide.

As used herein, a “broad spectrum protection” indicates that the sunscreen not only provides ultraviolet B (UVB) protection but also provides ultraviolet A (UVA) protection and meets US FDA standards for ‘broad spectrum’.

### Lip Balm Composition

A lip balm composition disclosed herein comprises a sunscreen and a lip balm base. The sunscreen can include a physical sunscreen. For example, the sunscreen can include a physical sunscreen in an amount of at least 60 wt %, or at least 70 wt %, or at least 80 wt %, or at least 90 wt %. In a particular embodiment, the sunscreen can include a physical sunscreen in an amount of at least 95 wt %, or at least 99 wt %, or even 100 wt %, based on a total weight of the sunscreen in the lip balm composition. In a particular embodiment, the sunscreen consists of a physical sunscreen. In a particular embodiment, the sunscreen does not contain a chemical sunscreen.

In an embodiment, the physical sunscreen can include a metal oxide. In a particular embodiment, the physical sunscreen can include a zinc oxide. In a particular embodiment, the physical sunscreen does not contain titanium dioxide. In a more particular embodiment, the physical sunscreen does not contain any metal oxide other than a zinc oxide.

In principle, as the amount of sunscreen is increased, the protection against harmful ultraviolet rays also increases. Accordingly, it would be advantageous to include an increased level of sunscreen in the lip balm composition. However, maintaining an increased level of sunscreen would be expected to significantly diminish the structure and moisturizing characteristics of the lip balm, as well as leave an undesirable opaque white cast on the skin of the lip.

Surprisingly, the inventors have discovered that by adjusting the components of the lip balm, an increased level of sunscreen can be dispersed and maintained in the lip balm while maintaining its structure and moisturizing characteristics, and also minimizing the opaque white cast.

In a particular embodiment, the sunscreen is present in the lip balm composition in an amount of at least 14 wt %, or at least 16 wt %, or at least 18 wt %, or even at least 20 wt %. In a particular embodiment, the sunscreen is present in the lip balm composition in an amount of no greater than 50 wt %, or no greater than 45 wt %, or no greater than 40 wt %, or no greater than 35 wt %. In a particular embodiment, the sunscreen is present in an amount in a range of 14 to 50 wt %, or 16 to 45 wt %, or 18 to 40 wt %, or 20 to 35 wt %.

In a more particular embodiment, zinc oxide is present in the lip balm composition in an amount of at least 14 wt %, or at least 16 wt %, or at least 18 wt %, or even at least 20 wt %. In a particular embodiment, the sunscreen is present in the lip balm composition in an amount of no greater than 50 wt %, or no greater than 45 wt %, or no greater than 40 wt %, or no greater than 35 wt %. In a particular embodiment, the sunscreen is present in an amount in a range of 14 to 50 wt %, or 16 to 45 wt %, or 18 to 40 wt %, or 20 to 35 wt %. The USP Monograph approves zinc oxide paste up to 25 wt % and, in an embodiment, zinc oxide is present in an amount in a range of 14 to 25 wt %, or 16 to 25 wt %, or 18 to 25 wt %, or 20 to 25 wt %

In an embodiment, the physical sunscreen comprises a non-nano material. In an embodiment, the physical sunscreen does not contain a nano material. In an embodiment, the zinc oxide contains a non-nano zinc oxide. In an embodiment, the zinc oxide does not contain a nano zinc oxide. The zinc oxide can have an average particle size of at least 100 nm, or at least 125 nm, or at least 150 nm, or at least 175 nm, or at least 200 nm. The zinc oxide can have an average particle size of no greater than 1000 nm, or no greater than 800 nm, or no greater than 600 nm, or no greater than 400 nm. The zinc oxide can have an average particle size in a range of 100 to 1000 nm, or 150 to 800 nm, or 200 to 600 nm.

Despite the high loading of zinc oxide, the lip balm composition can maintain a solid stick structure. The solid stick structure can have sufficient robustness for commercial consumer use. A solid stick structure does not have sufficient robustness if light pressure causes the structure to bend, curve, break, not extend from container, or change shape so as to no longer retract back into container. In an embodiment, the solid stick structure can have sufficient hardness as measured according to a hardness test.

Despite the high loading of zinc oxide, the lip balm composition can surprisingly avoid leaving an opaque white cast on the lip skin. For example, the lip balm composition can avoid leaving an opaque white cast as measured on a colorimeter.

Despite the high loading of zinc oxide and the solid stick structure, the lip balm composition can maintain a soft, rich, smooth texture on application; prevent drying; and maintain moisturization and skin barrier function levels of the skin. Moisturization or hydration is defined based on the water content of the stratum corneum of the epidermis. This is determined by measurement of the electrical capacitance.

In an embodiment, the lip balm composition can provide protection against ultraviolet A radiation. In an embodiment, the lip balm composition can provide protection against ultraviolet B radiation. In an embodiment, the lip balm composition can provide protection against ultraviolet A radiation and ultraviolet B radiation. In an embodiment, the composition can provide broad spectrum protection, such as at least broad-spectrum SPF 25.

As discussed above, the lip balm composition is able to maintain a high loading of zinc oxide, while maintaining its structure, texture, and translucent coating, in part due to the components of the lip balm base.

In an embodiment the lip balm base can contain a wax, or a butter, or an oil, or an ester, or any combination thereof. In an embodiment, the lip balm base can contain at least two of the above ingredients, such as a wax and a butter, a wax and an oil, a wax and an ester, a butter and an oil, a butter and an ester, or an oil and an ester. In an embodiment, the lip balm base can contain at least a wax, a butter, and an oil; or a wax, an oil, and an ester; or a wax, a butter, and an ester; or a butter, an oil and an ester.

In a more particular embodiment, the lip balm base can contain each of a wax, a butter, an oil, and an ester. For example, the wax can be combined with a butter, an oil, and an ester to form a lip balm base that has a rich, soft, smooth feel when applied to the lips.

As discussed above, the lip balm base can contain a wax. The selection of the nature of the wax and its content can contribute to the structure and feel of the lip balm. Among other things, a wax can impart structural support and rigidity to the lip balm base when the lip balm base is formed into a stick structure. However, too much wax can take away from the rich, soft, smooth lip feel of the lip balm base composition. In an embodiment, the wax can be present in the lip balm base in an amount of no greater than 25 wt %, or no greater than 20 wt %, or no greater than 15 wt %, based on a total weight of the lip balm base. In an embodiment, the wax can be present in the lip balm base in an amount of at least 6 wt %, or at least 8 wt %, or at least 10 wt %, based on a total weight of the lip balm base. In an embodiment, the wax can be present in an amount in a range of 6 to 25 wt %, or 8 to 20 wt %, or 10 to 15 wt %, based on a total weight of the lip balm base.

In an embodiment, the lip balm base can contain a synthetic wax in an amount of no greater than 1 wt %, or no greater than 0.5 wt %, or no greater than 0.1 wt %, based on a total weight of the lip balm base. In an embodiment, the wax does not contain a synthetic wax. In an embodiment, the lip balm composition does not contain a synthetic wax.

In an embodiment, at least 60 wt %, or at least 70 wt %, or at least 80 wt %, or at least 90 wt %, or at least 95 wt %, or at least 99 wt % of the wax in the composition comprises a conventional wax. In a particular embodiment, 100% of the wax in the lip balm base comprises a conventional wax. In a particular embodiment the wax consists essentially of a conventional wax. In a more particular embodiment, the wax consists of a conventional wax. Even in the context of a natural wax, certain waxes may be avoided, such as a mammalian wax. In an embodiment, the wax does not contain a mammalian wax. In an embodiment, the lip balm composition does not contain a mammalian wax.

In an embodiment, the conventional wax can include a rice bran wax, a sunflower wax, a berry wax, a myrica fruit wax, a jojoba wax, a beeswax, a carnauba wax, a candelilla wax, or any combination thereof. In certain embodiments, it is preferable to minimize the use of beeswax in the composition. For example, the ratio of botanical wax to beeswax can be in a range of 90:10 to 70:30, such as 86:14 to 74:26, or 82:18 to 78:22, based on a total amount of botanical wax and beeswax.

In an embodiment, the wax can contain a beeswax in an amount of no greater than 30 wt %, or no greater than 25 wt %, or no greater than 20 wt %, based on a total weight of the wax. In an embodiment, the wax can contain a beeswax in an amount of at least 6 wt %, or at least 9 wt %, or at least 12 wt %, based on a total weight of the wax.

In an embodiment, the wax can contain a botanical wax in an amount of at least 65 wt %, or at least 70 wt %, or at least 75 wt %, based on a total weight of the wax. In an embodiment, the wax can contain a botanical wax in an amount of no greater than 90 wt %, or no greater than 86 wt %, or no greater than 82 wt %, based on a total weight of the wax.

In an embodiment the conventional waxes used consist essentially of a botanical wax in an amount of less than about 15 wt % and a beeswax in an amount of less than about 5 wt %, based on a total weight of the lip balm composition.

As discussed above, the lip balm base can contain a butter. A butter can contribute skin-conditioning properties to the composition. In certain embodiments, the butter can include a plurality of butters as each butter can contribute in different ways based on the source of the butter. In a particular embodiment, the butter can contain a natural butter, derived/obtained from natural sources. In a more particular embodiment, the butter can contain a botanical butter (as defined earlier).

In addition to emollient and moisturizing properties, a botanical butter can have a variety of other attributes depending on the botanical source of the butter. For example, butters may vary in their sensory feel and/or have particular components with desired functionalities such as components that enhance lip barrier function, enhance penetration, or have antioxidant properties or the like. Accordingly, a combination of butters can provide for modulation of lip feel (such as, for example, suppleness and/or moistness) of the composition as well as a source of natural ingredients with beneficial attributes (such as, for example, emollient, moisturizer, medicament, and/or antioxidant).

In an embodiment, the butter can include at least two butters. In a particular embodiment, the butter can include at least three butters. Suitable butters include but are not limited to mango seed butter, raspberry butter, avocado butter, shea butter, olive butter, kuku butter, monoi butter, peach butter, pistachio butter, coconut butter, cocoa butter, pomegranate butter, rose hip butter, sunflower butter, wheat germ butter, apricot butter, babassu butter, cupuacu butter, kokum butter, hazelnut butter, jojoba butter, sesame butter, soy butter, almond butter, meadowfoam seed butter, black current seed butter, cranberry butter and combinations thereof. In a particular embodiment, the butter can include a mango butter, a murumuru butter, a shea butter, or any combination thereof.

The butter content in the lip balm base can affect stick structure, in-use application properties, and aesthetic skin feel. The appropriate selection and content/amount of butter can positively affect aesthetics and functionality to deliver a smooth, soft, consistent application, after-feel, and product deposition on the lip skin. An inappropriate selection or content/amount of butter can negatively affect aesthetics by softening the product too much such that the stick is no longer structurally sound nor is it able to successfully be applied to the skin with the intended application resulting in an excessive and non-uniform application. Depending on the total number of butters used, the total amount of butter is typically about 1% to about 20%, based on a total weight of the lip balm composition. In an embodiment, the butter can be present in an amount of at least 1.5 wt %, or at least 2 wt %, or at least 2.5 wt %, based on a total weight of the lip balm composition. In an embodiment, the butter can be present in an amount of no greater than 10 wt %, or no greater than 7 wt %, or no greater than 5 wt %, based on a total weight of the lip balm composition. In an embodiment, the butter can be present in an amount of 1.5 to 10 wt %, such as 2 to 7 wt %, such as 2.5 to 5 wt %, based on a total weight of the lip balm composition.

As discussed above, the lip balm base can contain an oil. Similar to the butter content, the oil content in the lip balm base can affect stick structure, in-use application properties, aesthetics, and skin feel. The appropriate source and content of oil can positively affect aesthetics and functionality to deliver a smooth, soft, consistent application, after-feel, and product deposition on the lip skin with increased glide. An inappropriate source or content of oil can negatively affect aesthetics by softening the product too much such that the stick is no longer structurally sound or non-solid at room temperature, nor is it able to successfully be applied to the skin with the intended application resulting in an excessive and non-uniform application resulting in an undesirable after feel commonly described by consumers as ‘greasy’. Exemplary oils suitable for use in the composition included, but are not limited to, sunflower oil, coconut oil, castor oil, vegetable oil, corn oil, aloe vera oil, canola oil, soybean oil, jojoba oil, olive oil, babassu oil, avocado oil, apricot oil, meadowfoam seed oil, macadamia seed oil, oat kernel oil, palm seed oil, safflower oil, sandalwood oil, sesame oil, almond oil, wheat germ oil, cranberry oil, and combinations thereof. In an embodiment, the lip balm base contains a sunflower oil, a coconut oil, an argan oil, a safflower oil, an aloe oil, or any combination thereof.

In view of consumer preferences for naturally sourced, ingredient transparency, and clean ingredient sourcing, botanical sourced oils are preferred. Depending on the type and number of oils used, the lip balm base may include oil in amounts of about 5% to about 65%. In an embodiment, the lip balm base can contain an oil in an amount of no greater than 50 wt %, or no greater than 45 wt %, or no greater than 40 wt %, based on a total weight of the lip balm composition. In an embodiment, the lip balm base can contain an oil in an amount of at least 20 wt %, or at least 25 wt %, or at least 20 wt %, based on a total weight of the lip balm composition. The oil can be present in an amount in a range of 25 to 45 wt %, such as 28 to 42 wt %, such as 30 to 40 wt %, based on a total weight of the lip balm composition.

To obtain both the emollient properties and desired structure a jojoba ester may be used. Jojoba esters are lipid esters derived from Jojoba plants. Depending on the size and structure of a given ester, the physical properties may range from waxy to oily. In view of commercially available jojoba ester products such as Floraesters supplied by FloraTech Americas, 1151 North Fiesta Blvd., Gilbert, Ariz. 85233. It may be convenient to use a combination of jojoba ester products. For example, in an exemplary embodiment, a jojoba ester product with a melting point range of 47-51° C. may be used in combination with a jojoba ester product having a melting point range of 56-61° C.

Typically, the total amount of jojoba ester is about 5% to about 50% of the total composition. In an embodiment, the botanical lipid ester can be present in an amount of at least 8 wt %, or at least 10 wt %, or at least 12 wt %, based on a total weight of the lip balm composition. In an embodiment, the botanical lipid ester can be present in an amount of no greater than 20 wt %, or no greater than 18 wt %, or no greater than 16 wt %, based on a total weight of the lip balm composition. In a particular embodiment, the botanical lipid ester can be present in an amount in a range of 8 to 20 wt %, such as 10 to 18 wt %, such as 12 to 16 wt %, based on a total weight of the lip balm composition.

The composition may further comprise one or more antioxidants. Antioxidants may protect the composition from oxidation (e.g., becoming rancid) and/or provide lip conditioning benefits and oxidation protection upon application to the lips.

In view of consumer preferences for naturally sourced products natural antioxidants such as tocopherols are preferred. Tocopherols, tocopheryl acetate, some botanical butters and green tea extracts are exemplary antioxidants suitable for use in the composition.

Optionally, the composition may further comprise a flavor. In view of consumer preferences, it is preferable to use a natural flavor material. Flavors are typically used in amounts of about 0.1% to 5%. Amounts may vary depending on the potency of the flavor and matrix in which the flavor is presented. Flavors may be presented in a botanical lipid matrix, for example.

Optionally, the composition may further comprise natural medicaments including, but not limited to, menthol, camphor, eucalyptus, salicylic acid, and derivatives of salicylic acid. Typically, medicaments would be added in amounts of less than about 3%. Amounts may vary depending on the potency of the medicament and the matrix in which the medicament is presented.

Optionally, colorant that imparts color to the composition may be included in the composition. The colorant should not be of an amount, particle size, and/or presented in a matrix that would permit transfer of colorant that imparts an opaque color to the lips during application. Natural colorants such as, for example, plant extracts, natural minerals, or carmine may be included.

Optionally, a sensate may be included in the composition. A sensate is a composition that initiates a sensory perception such as heating or cooling, for example, when contacted with the skin and/or lips. Exemplary sensates include, but are not limited to, mint extracts, cinnamon extract, and capsaicin. Preferably sensates are derived from natural sources. However, as sensates typically have high potency and accordingly may yield significant impact at low levels, synthetic sensates are within the scope of this invention.

Other beneficial agents known to one skilled in the art may likewise optionally be included in the composition. Aloe extracts and natural organic acids are exemplary of other beneficial agents. Natural organic acids including alpha-hydroxy acids may act as exfoliants, for example. Lactic acid is an exemplary alpha-hydroxy acid. Preferably any other beneficial agents are derived from botanical sources.

The lip balm base can be present in an amount of at least 50 wt % by weight of the lip balm composition. In a particular example, the lip balm base can be present in an amount of at least 54 wt %, or at least 58 wt %, or at least 62 wt %. The lip balm base can be present in an amount of no greater than 80 wt %, or no greater than 76 wt %, or no greater than 74 wt %. In a more particular embodiment, the lip balm base can be present in an amount in a range of 70 to 75 wt %.

### Method

A method disclosed herein can include a method of forming a lip balm composition. The method can include preparing a lip balm base and dispersing a sunscreen in the lip balm base to form a lip balm composition.

The step of preparing a lip balm base can include preparing it as a molten liquid mixture using one or more of the components discussed above and pouring into containers that form the stick shape as the molten mixture cools to a semi-solid state.

In one embodiment, the wax, butter, and oil are heated sufficiently to form a molten composition and then combined with the jojoba esters with mixing. As overheating may be detrimental to some butters, the temperature of the molten mixture is reduced and/or adjusted to not exceed 160° F. prior to the addition of the butters.

Preferably the temperature of the mixture is held in the range of 140-160° F. during any additional mixing steps subsequent to the addition of the butters. Additional mixing steps may include, for example, addition of antioxidants and/or flavors.

Upon combination of all ingredients, the molten composition is placed in a stick forming container or containers and allowed to solidify. Optionally, once placed in the container, the forming stick may be subjected to one or more heating and cooling cycles as it solidifies to optimize formation of the stick. Optionally, the stick-forming container may be a dispensing tube.

Alternatively, the molten composition may be filled into a mold and allowed to solidify in the mold to form a stick. Once formed, the stick is removed from the mold and placed in a dispensing container.

Typically, suitable dispensing containers comprise an elevator portion which permits extending the stick from the dispensing container to facilitate application to the user's lips and retraction of the stick back into the container for storage.

The step of dispersing sunscreen in the lip balm base can include establishing uniform dispersion of the zinc oxide through mixing capabilities that minimize the incorporation of air into the composition.

A method disclosed herein can include a method of applying sunscreen. The method can include obtaining a lip balm composition comprising sunscreen and applying the lip balm composition to a lip of a user.

### LIST OF EMBODIMENTS

### Embodiment 1

A composition comprising lip balm base and a sunscreen dispersed within the lip balm base, the sunscreen comprising a physical sunscreen and the physical sunscreen being present in the composition in an amount of at least 16 wt % based on a total weight of the composition, wherein the composition is a lip balm composition having a solid stick structure.

### Embodiment 2

A composition comprising a lip balm base and a sunscreen dispersed within the lip balm base, the sunscreen comprising a physical sunscreen and the physical sunscreen being present in the composition in an amount of at least 16 wt % based on a total weight of the lip balm composition, wherein the lip balm composition maintains moisturization on a lip of a user for at least 8 hours after it is applied to the lip of the user.

### Embodiment 3

A composition comprising a lip balm base and a sunscreen dispersed within the lip balm base, the sunscreen comprising a physical sunscreen and the physical sunscreen being present in the composition in an amount of at least 16 wt % based on a total weight of the lip balm composition, wherein, when applied to a user, the lip balm composition does not leave an opaque white cast on a lip of the user.

### Embodiment 3

The composition, wherein the physical sunscreen is present in the composition in an amount of at least 18 wt %, at least 20 wt %, at least 22 wt %, or at least 24 wt %.

### Embodiment 4

The composition of any one of the preceding embodiments, wherein the physical sunscreen comprises a zinc oxide.

### Embodiment 5

The composition of any one of the preceding embodiments, wherein the physical sunscreen does not contain any titanium dioxide.

### Embodiment 6

The composition of any one of the preceding embodiments, wherein the physical sunscreen does not contain any metal oxide other than a zinc oxide.

### Embodiment 7

The composition of any one of the preceding embodiments, wherein the physical sunscreen does not contain a sunscreen other than zinc oxide.

### Embodiment 8

The composition of any one of the preceding embodiments, wherein the physical sunscreen consists essentially of zinc oxide.

### Embodiment 9

The composition of any of the preceding embodiments, wherein the physical sunscreen makes up at least 60 wt %, or at least 70 wt %, or at least 80 wt %, or at least 90 wt % of the sunscreen in the composition, based on a total weight of the sunscreen in the composition.

### Embodiment 10

The composition of any of the preceding claims, wherein the physical sunscreen makes up at least 95 wt %, or at least 99 wt %, or even 100 wt % of the sunscreen in the composition, based on a total weight of the sunscreen in the composition.

### Embodiment 11

The composition of any one of the preceding embodiments, wherein the physical sunscreen makes up all of the sunscreen in the composition.

### Embodiment 12

The composition of any one of the preceding embodiments, wherein the composition comprises 0 to 1 wt %, or 0 to 0.1 wt %, or 0 to 0.01 wt % of a chemical sunscreen.

### Embodiment 13

The composition of any one of the preceding embodiments, wherein the zinc oxide is present in the composition in an amount of at least 18 wt %, at least 20 wt %, at least 22 wt %, or at least 24 wt %, based on a total weight of the composition.

### Embodiment 14

The composition of any one of the preceding embodiments, wherein the composition provides protection against ultraviolet A radiation.

### Embodiment 15

The composition of any one of the preceding embodiments, wherein the composition provides protection against ultraviolet B radiation.

### Embodiment 16

The composition of any one of the preceding embodiments, wherein the composition provides protection against ultraviolet A radiation and ultraviolet B radiation.

### Embodiment 17

The composition of any one of the preceding embodiments, wherein the composition provides broad spectrum protection, such as at least broad-spectrum SPF 25.

### Embodiment 18

The composition of any one of the preceding embodiments, wherein the mineral sunscreen comprises a non-nano material.

### Embodiment 19

The composition of any one of the preceding embodiments, wherein the mineral sunscreen does not contain a nano material.

### Embodiment 20

The composition of any one of the preceding embodiments, wherein the zinc oxide is a non-nano zinc oxide.

### Embodiment 21

The composition of any one of the preceding embodiments, wherein the zinc oxide has an average particle size of at least 100 nm, or at least 125 nm, or at least 150 nm, or at least 175 nm, or at least 200 nm.

### Embodiment 22

The composition of any one of the preceding embodiments, wherein the zinc oxide has an average particle size of no greater than 1000 nm, or no greater than 800 nm, or no greater than 600 nm, or no greater than 400 nm.

### Embodiment 23

The composition of any one of the preceding embodiments, wherein the composition does not contain oxybenzone, or avobenzone, or octisalate, or octocrylene, or homosalate, or octinoxate, or aminobenzoic acid, or trolamine salicylate, or any combination thereof.

### Embodiment 24

The composition of any one of the preceding embodiments, wherein the composition does not contain any chemical sunscreen.

### Embodiment 25

The composition of any one of the preceding embodiments, wherein the moisturizer comprises a botanical butter, or a botanical butter, or a botanical oil, or a natural wax, or a lip ester, or any combination thereof.

### Embodiment 26

The composition of any one of the preceding embodiments, wherein the lip balm base comprises a botanical butter, a botanical oil, a natural wax, and a botanical lipid ester.

### Embodiment 27

The composition of any one of the preceding embodiments, wherein the composition comprises a botanical butter.

### Embodiment 28

The composition of any one of the preceding embodiments, wherein the botanical butter comprises a shea butter, a mango butter, a murumuru butter, or any combination thereof.

### Embodiment 29

The composition of any one of the preceding embodiments, wherein the composition comprises the botanical butter in an amount of 1.5 to 7 wt %, such as 2 to 6 wt %, such as 2.5 to 5 wt %, based on a total weight of the composition.

### Embodiment 30

The composition of any one of the preceding embodiments, wherein the lip balm base comprises a botanical oil.

### Embodiment 31

The composition of any one of the preceding embodiments, wherein the botanical oil comprises a sunflower seed oil, a coconut oil, an argan tree oil, a safflower seed oil, or any combination thereof.

### Embodiment 32

The composition of any one of the preceding embodiments, wherein the composition comprises the botanical oil in an amount of 25 to 45 wt %, such as 28 to 42 wt %, such as 30 to 40 wt %.

### Embodiment 33

The composition of any one of the preceding embodiments, wherein the lip balm base comprises a botanical lipid ester.

### Embodiment 34

The composition of any one of the preceding embodiments, wherein the botanical lipid ester comprises a jojoba ester.

### Embodiment 35

The composition of any one of the preceding embodiments, wherein the composition comprises a botanical lipid ester in an amount of 8 to 20 wt %, such as 10 to 18 wt %, such as 12 to 16 wt %.

### Embodiment 36

The composition of any one of the preceding embodiments, wherein the composition comprises a jojoba ester in an amount of 8 to 20 wt %, such as 10 to 18 wt %, such as 12 to 16 wt %.

### Embodiment 37

The composition of any one of the preceding embodiments, wherein the lip balm base comprises a natural wax.

### Embodiment 38

The composition of any one of the preceding embodiments, wherein the natural wax comprises a non-mammalian wax.

### Embodiment 39

The composition of any one of the preceding embodiments, wherein the natural wax consists of a non-mammalian wax.

### Embodiment 40

The composition of any one of the preceding embodiments, wherein the natural wax comprises a candelilla wax, a beeswax, a carnauba wax, or any combination thereof.

### Embodiment 41

The composition of any one of the preceding embodiments, wherein the composition comprises a natural wax in an amount of 6 to 19 wt %, such as 8 to 17 wt %, such as 10 to 15 wt %.

### Embodiment 42

A container comprising a composition, the composition comprising the composition of any one of the preceding embodiments.

### Embodiment 43

The container of Embodiment 42, wherein the container contains the composition in a stick shape.

### Embodiment 44

The container of any one of Embodiments 42 and 43, wherein the container comprises a dispensing tube.

### Embodiment 45

The container of Embodiment 44, wherein the composition is able to extend from the container and retract back into the container.

### Embodiment 46

The container of Embodiment 45, wherein the container comprises a mechanism that can dispense the composition so that it extends from the container and retract the composition back into the container.

### Embodiment 47

The container of Embodiment 46, wherein the mechanism is a rotary mechanism.

### Embodiment 48

A method of making a composition, comprising:
dispersing a mineral sunscreen in a liquid lip balm formulation to form a liquid mixture,
solidifying the liquid mixture to form a stick lip balm comprising the dispersed mineral sunscreen,
wherein the stick lip balm comprises the dispersed mineral sunscreen in an amount of at least 16 wt % based on a total weight of the stick lip balm.

### Embodiment 49

A method of applying a lip balm composition to a user, comprising:
dispensing the lip balm composition from a container so that it extends from the container,
applying the lip balm composition on a lip of the user, and
retracting the lip balm composition back into the container,
wherein the lip balm composition is the composition of any one of Embodiments 1 to 41.

### Embodiment 50

The method of Embodiment 49, wherein the composition maintains its shape as it is retracted into the container.

### Examples

A randomized, controlled, open label clinical study in healthy adult volunteers was designed to evaluate the 8-hour moisturization efficacy of Sample 1 using corneometry. The study duration was 11 days.

Skin hydration (‘moisture’) was assessed before and after a single treatment with Sample 1 (2, 4, 6, and 8 hrs. post-treatment), compared to ‘no treatment’ as control. The formulation for Sample 1 is provided below in Table 1.

TABLE 1

| Component | Amount |
|---|---|
| Zinc Oxide Dispersion | 31% w/w dispersion (20.5% w/w zinc oxide) |
| Waxes | 12% w/w |
| Oils | 38% w/w |
| Butters | 4% w/w |
| Lipid Ester | 13% w/w |
| Vitamins, Tocopherols, | Remaining |
| and Flavors | |

The zinc oxide dispersion is in caprylic/capric triglyceride, Grant Industries.

The waxes include beeswax, candelilla, and carnauba waxes.

The oils include sunflower, safflower, aloe, argan, and coconut oils.

The butter include mango, murumuru, and shea butters.

The lipid ester includes jojoba lipid ester.

A 1-week (7 days±2 days) skin conditioning phase preceded the test day (Visit 2), during which study participants used only the soap provided for personal washing. No other products were applied to the skin of the test sites for the duration of the study. Each participant had 2 test sites delineated on the skin of their volar forearm (one per arm). Eligible participants were assigned to treatment based on the randomization schedule at the start of the test day (Visit 2).

Assignment of study treatments followed a randomization schedule. The sites treated with Sample 1 and untreated control sites were randomized between the right and left volar forearms in accordance with the computer-generated randomization table. The randomization table was generated prior to the study start date using analysis software SAS Version 9.4. The statistician responsible for data analysis did not have access to the randomization code until after the database had been locked.

Corneometer readings were recorded and statistical analysis was performed. Given the likely visual/textural differences between the treated and untreated test sites, the corneometer operator was not considered blinded. To help minimize bias, the corneometer operator did not participate in treatment application and the two technicians responsible for treatment application were not present for, and did not influence, the corneometer assessments. In addition, the randomization table and the seed were kept in a secure location; the statistician responsible for data analysis remained blinded to the treatment allocation until after the database had been locked. After the database lock, the statistician unblinded the study by assigning the treatment codes for the analysis.

Up to 10 participants were sequestered in a temperature/humidity monitored test room on each test day; the temperature and relative humidity (RH) were maintained at 21±2° C. and 50±10% RH, respectively, for the duration of each test day.

Corneometer values for the primary analysis population are provided in Appendix III and are summarized for each test site at each assessment time point (pre-treatment (baseline) and 2-, 4-, 6- and 8-hours post-treatment) in Table 2. A plot of the corneometer data is presented in FIG. 1. The higher the corneometer reading, the more hydrated (‘moist’) the skin. Baseline values were similar for the treated and untreated sites (23.5 and 24.2, respectively).

TABLE 2

| | Baseline | 2 Hours | 4 Hours | 6 Hours | 8 Hours |
|---|---|---|---|---|---|
| Treated Site | | | | | |
| (Sample 1) | | | | | |
| N | 36.0 | 36.0 | 36.0 | 36.0 | 36.0 |
| Raw Mean | 23.5 | 26.0 | 25.2 | 25.6 | 24.6 |
| SD | 5.7 | 7.2 | 6.7 | 8.7 | 6.6 |
| Median | 23.1 | 24.9 | 25.4 | 24.5 | 24.8 |
| Minimum | 12.1 | 12.2 | 11.3 | 11.2 | 12.2 |
| Maximum | 37.7 | 53.6 | 46.6 | 64.7 | 46.0 |
| Untreated Site | | | | | |
| (Control) | | | | | |
| N | 36.0 | 36.0 | 36.0 | 36.0 | 36.0 |
| Raw Mean | 24.2 | 23.6 | 24.1 | 24.2 | 23.9 |
| SD | 6.0 | 5.7 | 5.7 | 5.8 | 5.7 |
| Median | 25.0 | 24.5 | 25.2 | 25.3 | 25.5 |
| Minimum | 11.5 | 11.4 | 11.0 | 9.5 | 13.5 |
| Maximum | 38.0 | 40.0 | 40.6 | 41.0 | 39.2 |

Based on the results in Table 2, there was no decrease in moisturization levels when comparing the treated site using Sample 1 and the untreated site. Surprisingly, there was actually an increase in maximum moisturization levels when comparing the treated site baseline to the treated site over time, and when comparing the treated site over time to the untreated site over time. The results show the surprising effect that high loading of zinc oxide in Sample 1 does not dry out the skin, but instead maintains or even improves moisturization levels of the skin.

1-50. (canceled)

51. A composition comprising a lip balm base and a sunscreen dispersed within the lip balm base, the sunscreen comprising a physical sunscreen present in the composition in an amount of at least 16 wt % based on a total weight of the composition, wherein the composition has at least one of the following characteristics:
the composition is a lip balm composition having a solid stick structure;
the composition maintains moisturization on a lip of a user for at least 8 hours after it is applied to the lip of the user; or
the composition does not leave an opaque white cast on a lip of the user.

52. The composition of claim 51, wherein the physical sunscreen comprises a zinc oxide or consists essentially of a zinc oxide.

53. The composition of claim 51, wherein the physical sunscreen is free of titanium oxide.

54. The composition of claim 51, wherein the physical sunscreen comprises a zinc oxide and no other metal oxides.

55. The composition of claim 51, wherein the physical sunscreen is present in the composition in an amount of at least 18 wt %, at least 20 wt %, at least 22 wt %, at least 24 wt %, at least 60 wt %, at least 70 wt %, at least 80 wt %, at least 90 wt %, at least 95 wt %, at least 99 wt %, or 100 wt % of the sunscreen in the composition, based on the total weight of the composition.

56. The composition of claim 51, wherein the physical sunscreen makes up at least 60 wt %, or at least 70 wt %, or at least 80 wt %, or at least 90 wt % of the sunscreen in the composition, based on a total weight of the sunscreen in the composition.

57. The composition of claim 51, wherein the composition comprises 0 wt % to 1 wt %, or 0 wt % to 0.1 wt %, or 0 wt % to 0.01 wt % of a chemical sunscreen.

58. The composition of claim 51, wherein zinc oxide is present in the composition in an amount of at least 18 wt %, at least 20 wt %, at least 22 wt %, or at least 24 wt %, based on a total weight of the composition.

59. The composition of claim 58, wherein the zinc oxide has an average particle size of at least 100 nm, or at least 125 nm, or at least 150 nm, or at least 175 nm, or at least 200 nm, and wherein the zinc oxide has an average particle size of no greater than 1000 nm, or no greater than 800 nm, or no greater than 600 nm, or no greater than 400 nm.

60. The composition of claim 51, wherein the composition provides protection against ultraviolet A radiation or ultraviolet B radiation.

61. The composition of claim 51, wherein the composition does not contain oxybenzone, or avobenzone, or octisalate, or octocrylene, or homosalate, or octinoxate, or aminobenzoic acid, or trolamine salicylate, or any combination thereof.

62. The composition of claim 51, wherein the moisturizer or the lip balm base comprises a botanical butter, a botanical oil, a natural wax, a lipid ester, or any combination thereof.

63. The composition of claim 51, wherein the composition comprises botanical butter in an amount of 1.5 to 7 wt %, based on a total weight of the composition.

64. The composition of claim 51, wherein the composition comprises botanical oil in an amount of 25 to 45 wt % or a botanical lipid ester in an amount of 8 wt % to 20 wt %.

65. The composition of claim 51, wherein the composition comprises a jojoba ester in an amount of 8 to 20 wt %.

66. The composition of claim 51, wherein the composition comprises a natural wax in an amount of 6 to 19 wt %, such as 8 to to 17 wt %, such as 10 to 15 wt %, and wherein the natural wax comprises a candelilla wax, a beeswax, a carnauba wax, or any combination thereof.

67. A container comprising the composition of claim 51, wherein the composition is formed in a stick shape, and wherein the container comprises a dispensing tube having a rotary mechanism adapted to extend the composition from and retract the composition back into the container.

68. A method of making a composition, comprising: dispersing a mineral sunscreen in a liquid lip balm formulation to form a liquid mixture, solidifying the liquid mixture to form a stick lip balm comprising the dispersed mineral sunscreen, wherein the stick lip balm comprises the dispersed mineral sunscreen in an amount of at least 16 wt % based on a total weight of the stick lip balm.

69. A method of applying a lip balm composition to a user, comprising: dispensing the lip balm composition from a container so that it extends from the container, applying the lip balm composition on a lip of the user, and retracting the lip balm composition back into the container, wherein the lip balm composition is the composition of claim 1.

70. The method of claim 69, wherein the composition maintains its shape as it is retracted into the container.
